(19) United States
(12) Plant Patent Application Publication
(10) Pub. No.: US 2014/0245501 P1
(43) Pub. Date: Aug. 28, 2014

(54) Avocado tree named 'Flavia'

(50) ;

(71) Applicant: AGROFRUTICOLA SAN BERNARDO DE MALLARAUCO LIMITADA, (US)

(72) Inventor: Alfredo Schiappacasse Macchiavello, Santiago (CL)

(73) Assignee: AGROFRUTICOLA SAN BERNARDO DE MALLARAUCO LIMITADA, Santiago (CL)

(21) Appl. No.: 13/815,426

(22) Filed: Feb. 28, 2013

(51) Int. Cl. A01H 5/00

(52) U.S. Cl. PLT/200

(57) ABSTRACT

The ‘Flavia’ avocado is notable for its larger fruit size and earlier fruit maturity for harvesting than ‘Hass’.

### LATIN NAME OF THE GENUS AND SPECIES

Persea americana Mill.

### VARIETAL DENOMINATION

‘Flavia’

### BACKGROUND OF THE INVENTION

‘Flavia’ is a new and distinct variety of avocado tree Persea americana Mill. The variety was first discovered in 1998 by Alfredo Schiapacasse Macchiavello, who noted a tree that produced bigger fruit in his compact ‘Hass’ cultivated avocado orchard in the Mallarauco Valley in Chile. The tree is located in the orchard called Ensenada, in the plot called El Marco.

### SUMMARY OF THE INVENTION

The ‘Flavia’ discovered tree is presumed to be a mutated bud (a whole tree mutation) from ‘Hass’. Budwood from the originally discovered tree was grafted into new trees, which were planted in 2001 as a non-propagation trial, and has since been observed to remain true to type over successive asexually propagated generations.

The tree was discovered after observing through several years that the fruit size was consistently larger than the other ‘Hass’ trees from the orchard. After several evaluations, it has been determined that the tree consistently produces larger fruit than ‘Hass’. Moreover, ‘Flavia’ reaches minimum dry matter level (23% DM), and thus can be harvested, four to five weeks earlier than regular ‘Hass’.

### BRIEF DESCRIPTION OF THE PHOTOGRAPHS

The accompanying photographs show specimens of the tree and plant parts of the new ‘Flavia’ variety.

FIG. 1 is a photograph of the originally discovered ‘Flavia’ mother tree;

FIG. 2 is a photograph depicting fruit and leaves of ‘Flavia’;

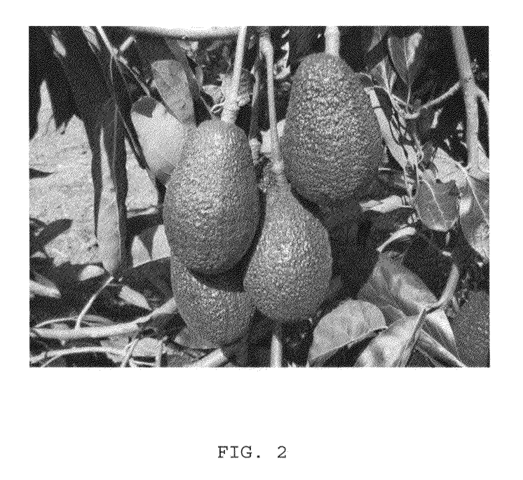

FIG. 3 is a photograph depicting fruit and leaves next to a hand holding a pen for size reference;

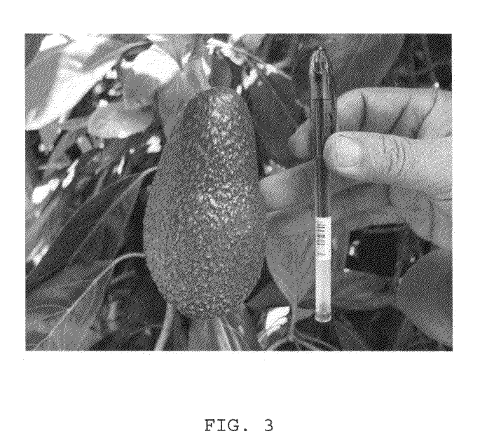

FIG. 4 is a photograph of four ‘Flavia’ fruit above four typical ‘Hass’ fruit, illustrating the enlarged nature of the ‘Flavia’ fruit; and

FIG. 5 is a photograph depicting sectioned fruit of the ‘Flavia’ above regular ‘Hass’ fruit.

### DETAILED BOTANICAL DESCRIPTION OF THE VARIETY

The following detailed botanical description is based on observations of a 2007 report based upon ‘Flavia’ trees planted in 2001. The grower of ‘Flavia’ at times refers to it by the code names GAMA 4022 or Andes 3. The 2007 report comprised a four-year study evaluating and comparing the ‘Flavia’ trees to similar trees grafted with ‘Hass’. The trees were planted in Sep. 2001, as a non-propagation trial in Huerto California in Quillota, Chile. Budwood from the mother ‘Flavia’ tree was granted into new trees which were planted as part of the non-propagation trial.

Aside from the fact that the ‘Flavia’ produces larger fruit than the regular ‘Hass’, and reaches dry minimum matter level four to five weeks earlier than regular ‘Hass’, it is otherwise nearly identical in color and appearance as to regular ‘Hass’ trees.

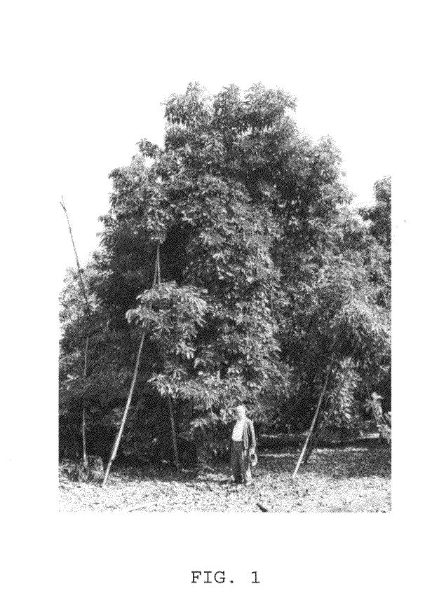

More particularly, the tree presents a vigorous upright growth, as illustrated in FIG. 1. The young shoots are reddish in color. The young shoot lenticels are purple in color. The anise aroma of the leaf blade is absent or very weak, similar to ‘Hass’. The inflorescence flowering type is similar to ‘Hass’ Type A.

With respect to the fruit, the surface of the mature fruit is rough, similar to ‘Hass’. The thickness of the pedicel compared to the peduncle, at the junction, is thicker. The pedicel length is medium. The pedicel shape is cylindrical. There is no “nail head” pedicel. The thickness of the ripe fruit skin is moderately thick, similar to ‘Hass’. Also similar to ‘Hass’, the ripe fruit color is a dark purple or black.

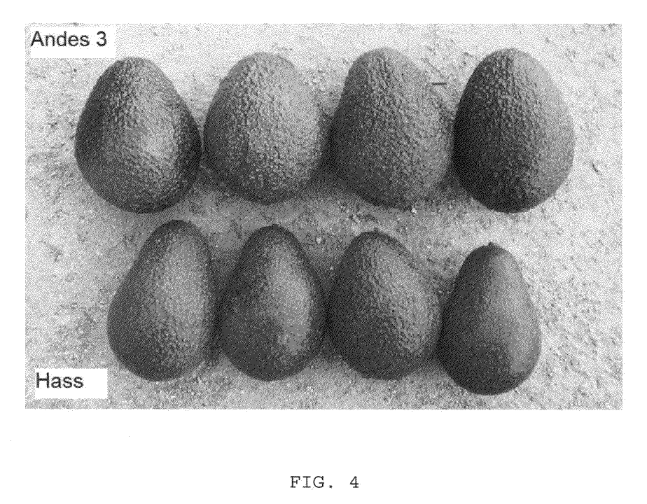

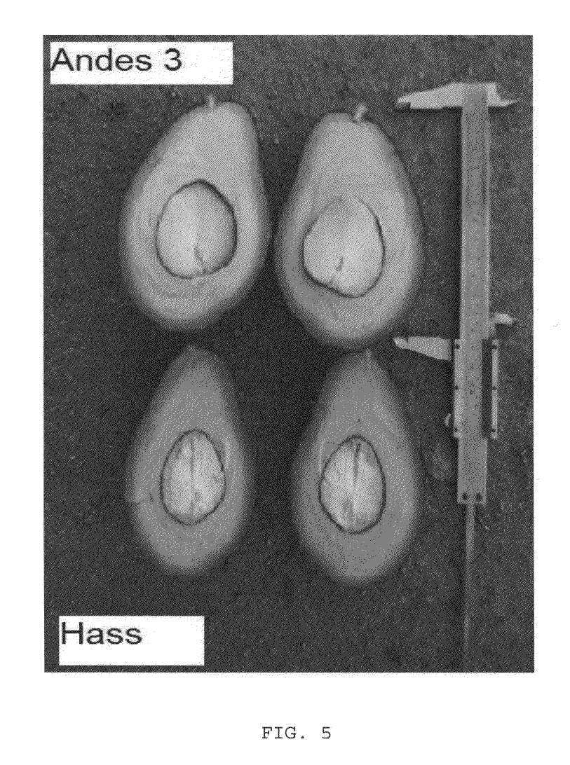

However, unlike ‘Hass’, the shape in the longitudinal section or lateral view of the seed is generally ovate, as illustrated in FIG. 5. With respect to the fruit shape, the polar/equatorial relation of the fruit is higher in ‘Flavia’ than in ‘Hass’, as seen in FIGS. 4 and 5, making the fruit more elongated than ‘Hass’. The seed of the ‘Flavia’ fruit is bigger than that of ‘Hass’, as shown in FIG. 5, and the seed/pulp relation is greater in ‘Flavia’ than in ‘Hass’. As can be seen in FIG. 4, the fruit of ‘Flavia’ is much larger than that of regular ‘Hass’.

Another distinctive feature of ‘Flavia’ is that the fruit matures for harvesting four to five weeks earlier than regular ‘Hass’. The time of fruit maturity for harvesting, when the fruit reaches minimum dry matter level (23% DM) is medium to late, between ‘Fuerte’ and ‘Hass’.

What is claimed is:

1. A new and distinct variety of avocado tree, substantially as illustrated and described herein.
